(19) United States
(12) Patent Application Publication
(10) Pub. No.: US 2022/0059232 A1
(43) Pub. Date: Feb. 24, 2022

(54) System, Method, and Computer Program Product for Monitoring Individuals in an Environment to Determine an Onset of Infection

(71) Applicant: ONDO Systems, Inc., Redwood City, CA (US)

(72) Inventors: Suguru Nishioka, San Francisco, CA (US); Ivan Goering, Santa Clara, CA (US); Marc Baum, San Jose, CA (US)

(21) Appl. No.: 17/410,357

(22) Filed: Aug. 24, 2021

Related U.S. Application Data
(60) Provisional application No. 63/069,629, filed on Aug. 24, 2020.

(51) Int. Cl. G16H 50/30; G16H 40/67; H04W 4/80

(52) U.S. Cl. CPC G16H 50/30; H04W 4/80; G16H 40/67

(57) ABSTRACT

Provided are computer-implemented methods for monitoring individuals in an environment to determine an onset of infection which includes receiving data associated with one or more measurements obtained by one or more sensors of a sensor band and determining whether the one or more measurements obtained by the one or more sensors of the sensor band satisfies one or more conditions of a non-standard state, wherein the non-standard state is associated with an individual at risk for infection. Such methods may also include providing an indication that an individual associated with the sensor band is at risk for infection. Systems and computer program products are also provided.

### CROSS REFERENCE TO RELATED APPLICATION

This application claims the benefit of U.S. Provisional Patent Application No. 63/069,629 filed Aug. 24, 2020, the contents of which are incorporated herein in its entirety by reference.

### BACKGROUND

### Field of the Invention

This disclosure relates generally to monitoring individuals in an environment to determine an onset of infection and, in some non-limiting embodiments or aspects, to systems, methods, and computer program products for monitoring individuals in an environment to determine infections, onset of infections, and temperature pattern for individuals and/or groups.

### Description of Related Art

Individuals (e.g., employees, long-term care patients, and/or the like) may be associated with an environment (e.g., an office building, a long-term care facility, and/or the like). These individuals may come into contact with infectious individuals (e.g., individuals infected with a pathogen such as a virus, etc.) or may otherwise become infected (e.g. from a bacterial growth, etc.). Often, these individuals are not identified as infected until the individuals are visibly present and/or become sufficiently self-aware and able to communicate symptoms (e.g., fever, headache, drowsiness, aches, and/or the like). Individuals may be symptomatic without realizing and/or without communicating their infection status to caregivers. To identify whether these individuals are infected before the individuals are aware of symptoms they are presenting, the individuals may be screened using a thermometer, rapid tests, and/or the like.

However, the above-noted techniques for identifying whether an individual is infected, at risk for infection, or in close contact with infected individuals, may not identify such individuals before they present symptoms and/or are aware of the symptoms they present. For example, use of a thermometer may be intermittent (e.g., every day) and individuals whose temperature increases throughout the day may not be identified until the following day when they are screened. Additionally or alternatively, the individuals may take medicine (e.g., acetaminophen) to address a symptom (e.g., a headache) that the individual is unaware may indicate they are infected. This may result in delays and/or foregoing identifying an individual as infected or at risk for infection.

### SUMMARY

Accordingly, disclosed are improved systems, methods, and computer program products for monitoring individuals, and collections or groups of individuals collectively, in an environment to determine whether the individuals, and or collections or groups of individuals, are infected and/or at risk of infection or other adverse health conditions.

According to some non-limiting embodiments or aspects, provided is a computer-implemented method, for monitoring individuals in an environment to determine whether the individuals are at risk for infection, the computer-implemented method comprising: receiving, with at least one processor, data associated with one or more measurements obtained by one or more sensors of a sensor band; determining, with at least one processor, whether the one or more measurements obtained by the one or more sensors of the sensor band satisfies one or more conditions of a non-standard state, wherein the non-standard state is associated with an individual at risk for infection; and providing, with at least one processor, an indication that an individual associated with the sensor band is at risk for infection.

According to some non-limiting embodiments or aspects, provided is a system, comprising at least one processor, programmed or configured to monitor individuals in an environment to determine whether the individuals are at risk for infection, and receive, with at least one processor, data associated with one or more measurements obtained by one or more sensors of a sensor band; determine, with at least one processor, whether the one or more measurements obtained by the one or more sensors of the sensor band satisfies one or more conditions of a non-standard state, wherein the non-standard state is associated with an individual at risk for infection; and provide, with at least one processor, an indication that an individual associated with the sensor band is at risk for infection.

According to some non-limiting embodiments or aspects, provided is a computer program product, comprising at least one non-transitory computer-readable medium including program instructions that, when executed by at least one processor, cause the at least one processor to: monitor individuals in an environment to determine whether the individuals are at risk for infection, and receive, with at least one processor, data associated with one or more measurements obtained by one or more sensors of a sensor band; determine, with at least one processor, whether the one or more measurements obtained by the one or more sensors of the sensor band satisfies one or more conditions of a non-standard state, wherein the non-standard state is associated with an individual at risk for infection; and provide, with at least one processor, an indication that an individual associated with the sensor band is at risk for infection.

Further embodiments or aspects are set forth in the following clauses:

Clause 1: A computer-implemented method, for monitoring individuals in an environment to determine whether the individuals are at risk for infection, the computer-implemented method comprising: receiving, with at least one processor, data associated with one or more measurements obtained by one or more sensors of a sensor band; determining, with at least one processor, whether the one or more measurements obtained by the one or more sensors of the sensor band satisfies one or more conditions of a non-standard state, wherein the non-standard state is associated with an individual at risk for infection; and providing, with at least one processor, an indication that an individual associated with the sensor band is at risk for infection.

Clause 2: The computer-implemented method of clause 1, wherein receiving the data associated with the one or more measurements obtained by the one or more sensors of the sensor band comprises: receiving the data associated with the one or more measurements obtained by the one or more sensors associated with the individual based on the sensor band transmitting the data associated with the one or more measurements obtained by the one or more sensors, wherein the sensor band transmits the data associated with the one or more measurements obtained by the one or more sensors by advertising the data associated with the one or more measurements to at least one gateway device.

Clause 3: The computer-implemented method of clauses 1-2, wherein transmitting the data associated with the one or more measurements obtained by the one or more sensors of the sensor band by advertising the data associated with the one or more measurements to at least one gateway device comprises: transmitting the data associated with the one or more measurements obtained by the one or more sensors of the sensor band by advertising the data associated with the one or more measurements from a Bluetooth low energy peripheral device included in the sensor band to the at least one gateway device.

Clause 4: The computer-implemented method of clauses 1-3, wherein receiving the data associated with the one or more measurements obtained by the one or more sensors of the sensor band comprises: receiving data associated with a temperature measurement of the individual; and receiving data associated with a temperature measurement of an environment in which the individual is located, and wherein determining whether the one or more measurements obtained by the one or more sensors of the sensor band satisfies one or more conditions of the non-standard state comprises: determining a core temperature of the individual based on the temperature measurement of the individual and the temperature measurement of the environment in which the individual is located; comparing the core temperature of the individual to a predetermined range of core temperatures corresponding to a high-temperature condition included in the one or more conditions of a non-standard state; and determining that the core temperature of the individual satisfies a predetermined range of core temperatures corresponding to the high-temperature condition included in the one or more conditions of the non-standard state, and wherein providing the indication that the individual is at risk for infection comprises: providing the indication that the individual is at risk for infection based on determining that the core temperature of the individual satisfies the predetermined range of core temperatures corresponding to the high-temperature condition included in the one or more conditions of the non-standard state.

Clause 5: The computer-implemented method of clauses 1-4, further comprising: receiving data associated with registration of the one or more sensors from the sensor band; registering the sensor band with the individual; and generating a profile corresponding to the individual based on the data associated with the one or more measurements obtained by one or more sensors of the sensor band.

Clause 6: The computer-implemented method of clauses 1-5, further comprising: determining that the one or more measurements obtained by the one or more sensors of the sensor band represents one or more trends; and determining whether the one or more trends indicate that the individual is at risk for infection, wherein providing the indication that the individual associated with the sensor band is at risk for infection comprises: providing the indication that the individual associated with the sensor band is at risk for infection based on determining that the trend indicates that the individual is at risk for infection.

Clause 7: The computer-implemented method of clauses 1-6, further comprising: determining that the one or more measurements obtained by the one or more sensors of the sensor band represents one or more trends; and comparing the one or more trends to the profile corresponding to the individual; and determining whether the one or more trends indicate that the individual is at risk for infection based on comparing the one or more trends to the profile corresponding to the individual, wherein providing the indication that the individual associated with the sensor band is at risk for infection comprises: providing the indication that the individual associated with the sensor band is at risk for infection based on determining that the one or more trends indicate that the individual is at risk for infection based on comparing the one or more trends to the profile corresponding to the individual.

The features and characteristics of the present disclosure, as well as the methods of operation and functions of the related elements of structures and the combination of parts and economies of manufacture, will become more apparent upon consideration of the following description with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification, the singular form of “a,” “an,” and “the” include plural referents unless the context clearly dictates otherwise.

### BRIEF DESCRIPTION OF THE DRAWINGS

Additional advantages and details are explained in greater detail below with reference to the non-limiting, exemplary embodiments that are illustrated in the accompanying figures, in which:

FIG. 1 is a diagram of non-limiting embodiments or aspects of an environment in which systems, devices, products, apparatus, and/or methods, described herein, may be implemented;

FIG. 2 is a diagram of non-limiting embodiments or aspects of components of one or more devices and/or one or more systems of FIG. 1;

FIG. 3 is a flowchart of non-limiting embodiments or aspects of a process for monitoring individuals in an environment to determine whether the individuals are at risk for infection;

FIG. 4 is a diagram of non-limiting embodiments or aspects of an environment in which systems, devices, products, apparatus, and/or methods, described herein, may be implemented; and

FIG. 5A-5D are illustrations of non-limiting embodiments or aspects of a system for monitoring individuals in an environment to determine an onset of infection.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosure as it is oriented in the drawing figures. However, it is to be understood that the disclosure may assume various alternative variations and step sequences, except where expressly specified to the contrary. It is also to be understood that the specific devices and processes illustrated in the attached drawings, and described in the following specification, are simply exemplary embodiments or aspects of the disclosure. Hence, specific dimensions and other physical characteristics related to the embodiments or aspects of the embodiments disclosed herein are not to be considered as limiting unless otherwise indicated.

No aspect, component, element, structure, act, step, function, instruction, and/or the like used herein should be construed as critical or essential unless explicitly described as such. In addition, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and transmits the processed information to the second unit. In some non-limiting embodiments or aspects, a message may refer to a network packet (e.g., a data packet and/or the like) that includes data.

As used herein, the term “system” may refer to one or more devices or combinations of devices such as, but not limited to, processors, servers, client devices, software applications, and/or other like components. In addition, reference to “a server” or “a processor,” as used herein, may refer to a previously-recited server and/or processor recited as performing a previous step or function, a different server and/or processor, and/or a combination of servers and/or processors. For example, as used in the specification and the claims, a first server and/or a first processor that are recited as performing a first step or function may refer to the same or different server and/or a processor recited as performing a second step or function.

As used herein, Bluetooth may refer to a protocol or communication system in which connections are made between a master and a slave. These connections are maintained until they are broken, either by deliberately disconnecting the two, or by the radio link (e.g., connection, etc.) becoming so poor that communications cannot be maintained—typically this occurs as the devices go out of range of each other. The connections in Bluetooth formed by pairing devices, such as by providing a handshake defined by a form of information registration for linking devices. By registering device information (pairing) between devices, they can connect. To use a Bluetooth device, you must first pair it with another Bluetooth device. Pairing is a bit like exchanging phone numbers. In some examples, the way in which Bluetooth devices make connections using frequencies (e.g., frequency hopping, frequency ranges, etc.). While frequency hopping reduces the effects of interference, it makes connecting devices more complicated. Once Bluetooth pairing has occurred two devices may communicate with each other. In existing systems, Bluetooth pairing is initiated manually by a device user. The Bluetooth link for the device is made visible to other devices that may then be paired. The Bluetooth pairing process is typically triggered automatically the first time a device receives a connection request from a device with which it is not yet paired. In order that Bluetooth pairing may occur, a code (e.g., password, passkey, etc.) has to be exchanged between the two devices. The code is shared by both Bluetooth devices. It is used to ensure that both users have agreed to pair with each other. A Bluetooth device looks for other Bluetooth devices in range: To be found by other Bluetooth devices, the first device, Device 1 must be set to discoverable mode—this will allow other Bluetooth devices in the vicinity to detect its presence and attempt to establish a connection.

As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, and/or the like.

As used herein, the term “computing device” may refer to one or more electronic devices that are configured to directly or indirectly communicate with or over one or more networks. A computing device may be a mobile or portable computing device, a desktop computer, a server, and/or the like. Furthermore, the term “computer” may refer to any computing device that includes the necessary components to receive, process, and output data, and normally includes a display, a processor, a memory, an input device, and a network interface. A “computing system” may include one or more computing devices or computers. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.). Further, multiple computers, e.g., servers, or other computerized devices, such as an autonomous vehicle including a vehicle computing system, directly or indirectly communicating in the network environment may constitute a “system” or a “computing system”.

As used herein, the terms “computing device”, “mobile device”, “client”, and “client device” may refer to one or more client-side devices or systems (e.g., remote from a data source) used to initiate, facilitate, or monitor a sensor event. As an example, a “client device” may refer to one or more health devices used by a patient care facility, one or more health computers used by a health monitoring system, one or more mobile devices used by a user, and/or the like. In some non-limiting embodiments or aspects, a client device may be an electronic device configured to communicate with one or more networks and initiate or facilitate communications. For example, a client device may include one or more computers, portable computers, laptop computers, tablet computers, mobile devices, cellular phones, wearable devices (e.g., watches, glasses, lenses, clothing, and/or the like), PDAs, and/or the like.

As used herein, the term “platform” may refer to one or more computing devices (e.g., processors, storage devices, servers, similar computer components, and/or the like) that communicate with client devices and/or other computing devices over a network (e.g., a public network, the Internet, a private network, and/or the like) and, in some examples, facilitate communication among servers and/or client devices. It will be appreciated that various other arrangements are possible. As used herein, the term “system” may refer to one or more computing devices or combinations of computing devices (e.g., processors, servers, client devices, software applications, components of such, and/or the like). Reference to “a device,” “a server,” “a processor,” and/or the like, as used herein, may refer to a previously-recited device, server, or processor that is recited as performing a previous step or function, a different server or processor, and/or a combination of servers and/or processors. For example, as used in the specification and the claims, a first server or a first processor that is recited as performing a first step or a first function may refer to the same or different server or the same or different processor recited as performing a second step or a second function.

As used herein, the term “supervised learning” may refer to one or more machine learning algorithms that start with known input variables (x) and an output variable (y), and learn a temperature monitoring function or pattern from the input to the output. The goal of supervised learning is to approximate the temperature function so that predictions can be made about new input variables (x) that can be used to predict the output variables (y) for that data. The process of a supervised algorithm learning from the training dataset can be thought of as a teacher supervising the learning process. The correct answers are known. The algorithm iteratively makes predictions on the training data and is corrected by the teacher. Learning stops when the algorithm achieves an acceptable level of performance. Supervised learning problems can be further grouped into regression problems and classification problems. Supervised learning techniques can use labeled (e.g., classified) training data with normal and outlier data, but are not as reliable because of the lack of labeled outlier data. For example, multivariate probability distribution based systems are likely to score the data points with lower probabilities as outliers. A regression problem is when the output variable is a real value, such as “dollars” or “weight”. A classification problem is when the output variable is a category, such as “red” and “blue” or “compliant” and “non-compliant”.

As used herein, the term “unsupervised learning” may refer to an algorithm which has input variables (x) and no corresponding output variables. The goal for unsupervised learning is to model the underlying structure or distribution in the data in order to learn more about the data. Unlike supervised learning, in unsupervised learning there are no correct answers and there is no teacher. Unsupervised learning algorithms are used to discover and present the interesting structure in the data. Unsupervised learning problems can be further grouped into clustering and association problems. A clustering problem is modeling used to discover the inherent groupings in a dataset, such as grouping customers by purchasing behavior. An association rule learning problem is where you want to discover rules that describe large portions of data, such as people that buy A also tend to buy B. Some examples of unsupervised learning algorithms are clustering and likelihood modeling.

As used herein, the term “training” may refer to a process of analyzing training data to generate a model (e.g., create a machine learning algorithm, a prediction model, a classification model, a segmentation model, etc.). For example, a training server uses machine learning techniques to analyze the training data to generate the model, often the training data includes numerous examples so that a robust model is generated to solve a problem for many variations present in the data. In some non-limiting embodiments or aspects, generating the model (e.g., based on training data from a variety of sources) is referred to as “training the model.” The machine learning techniques include, for example, supervised and/or unsupervised techniques, such as decision trees (e.g., gradient boosted decision trees), logistic regressions, artificial neural networks (e.g., convolutional neural networks), Bayesian statistics, learning automata, Hidden Markov Modeling, linear classifiers, quadratic classifiers, association rule learning, and/or the like. In some non-limiting embodiments or aspects, the model includes a prediction model that is specific to a particular geographic location, a particular client, a particular agent, a particular seller, a particular resource, and/or the like. Additionally or alternatively, the prediction model may be specific to a particular user (e.g., a user of a beacon band sending temperature readings to a cloud hosted monitoring platform, a corporate user monitoring one or more user environments for infected individuals, a user of a beacon band in a patient care facility, an aggregated group or subset of users of interest for temperature patterns, etc.). In some non-limiting embodiments or aspects, a training server generates one or more prediction models (e.g., one or more infection prediction models, one or more infection segmentations, etc.) for one or more operators of one or more accounts (e.g., one or more customer accounts, one or more patient care facility accounts, one or more employers, one or more educational facilities, one or more governmental agencies or departments, etc.), a particular group of customers, and/or the like.

The systems and methods of the disclosed systems are an improvement over a digital thermometer that does not communicate with any mobile and other connected devices and/or computational cloud infrastructure or a connected thermometer that pairs with a single companion device such as a smart phone. Such business-to-consumer (B2C) focused systems provide limited assistance to users and institutions which require routine monitoring for infection risks because they are not designed to be used 24/7 and cannot be deployed in bulk due to costs and system complexity. Non-connected thermometers create labor intensive workflows and documentation gaps. Connected B2C systems are not institutionally deployable and require outdated communication over a traditional phone communication. Required pairing to a phone necessitates each end user have a phone and individual user profile in an associated software application, which can be impractical in many situations, including in a caregiver facility. Such systems are inoperable or inefficient because they require the monitoring of multiple devices and/or paired phones that are each being used by a single user and need to be checked and monitored across multiple applications or platforms for just one user to be monitored.

In other systems, a reading is taken by a device stationed at an entrance to an industrial complex, such as a building. However, with only one reading, when a person is walking into a facility from outside, the conditions outside (e.g., hot or cold) can cause skin temperature to exhibit a hot or cold condition unrelated to core body temperature, causing anomalies such as a false positive or false negative. Such systems provide no ongoing monitoring once an individual has entered a building or for any individual permanently residing inside the environment, significantly limiting their efficacy.

In some existing systems, a band or wrist device may be available but not designed to provide a population management solution platform; users must pair to a phone and use the phone as a personal tracking device. Pairing to a phone will function as a system in institutional setup where several dozens of residents, patients, and/or workers must be monitored by several sets of caretaker, nursing and management teams as each phone must be present in close proximity to each paired thermometer. Pairing to a phone and maintaining a connection cause a need for extensive charging of both the device and phone, larger batteries, connectivity reliability issues, compromised capabilities due to highly limited platform specific protocols, and complicated applications and phone specific engineering requirements.

Other local area network communication protocols for wireless devices (e.g. ZigBee, Z-Wave, Wi-Fi) have connectivity or power consumption issues that do not facilitate or provide efficient support for devices which are physically moving around within the network or geospatial area. Such networks require persistent device communication, static device locations, paired communication protocols that inhibit quickly adding and removing devices from the network or scaling to manage larger populations, and significantly higher power consumption in the case of Wi-Fi.

The present disclosure solves these problems and has provides methods and systems with none of these constraints. It can handle many devices (e.g., 200 or more devices simultaneously) because they are not are not paired to phones or other devices, operate for extended service lives (several months up to years), and all device management is done at the cloud, not network level. Software and firmware updates can be pushed while users are moving anywhere in the environment. There are no hardware or communication protocol constraints on how many users can be simultaneously monitored by the system and be supported by the infrastructure.

In some non-limiting embodiments or aspects, beacon capable devices of the present disclosure include a new framework based on the capabilities of the devices to solve such problems.

Provided are improved systems, methods, and computer program products for monitoring individuals in an environment to determine whether the individuals are at risk for infection. In some non-limiting embodiments or aspects, systems, methods, or computer program products may include receiving, with at least one processor, data associated with one or more measurements obtained by one or more sensors of a sensor band; determining, with at least one processor, whether the one or more measurements obtained by the one or more sensors of the sensor band satisfies one or more conditions of a non-standard state, wherein the non-standard state is associated with an individual at risk for infection; and providing, with at least one processor, an indication that an individual associated with the sensor band is at risk for infection.

By virtue of implementation of the systems, methods, and computer program products described herein, individuals that are at risk for infection may be more accurately identified. For example, some embodiments described herein more efficiently identify infectious individuals and/or individuals at risk for infection than existing systems, in particular, efficiently and accurately tracking status of individuals and/or groups of individuals before the individuals present symptoms and/or are aware of the symptoms they present. In some examples, implementations described herein may continuously or at regular intervals throughout the day (e.g., every second, every minute, every hour, and/or the like) determine whether the temperature of an individual has increased. Additionally or alternatively, filling a gap in existing system, in some embodiments, individuals are more accurately identified as at risk for infection prior to and/or after the individuals take medicine to address symptoms that mask indications that the individual is at risk for infection. As a result of the embodiments described herein, delays in diagnosing illness are reduced when identifying an individual as infected or at risk for infection, and additionally, monitoring a herd of individuals identifies trends more accurately and sufficiently to avoid an outbreak in a community.

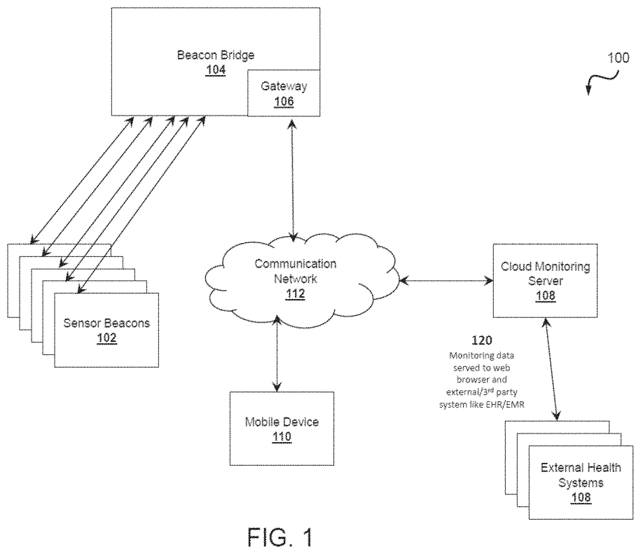

Referring now to FIG. 1, FIG. 1 is a diagram of an example temperature and infection monitoring system 100 for monitoring individuals in an environment to determine whether the individuals are at risk in which devices, systems, methods, and/or products described herein, may be implemented. As shown in FIG. 1, temperature and infection monitoring system 100 includes sensor beacons 102, beacon bridge 104, gateway 106, cloud monitoring server 108, mobile device 110, and communication network 112. Systems and/or devices of temperature and infection monitoring system 100 can interconnect via wired connections, wireless connections, or a combination of wired and wireless connections for early detection and population management of infectious diseases (e.g., COVID-19 virus, corona virus, influenza, bacterial infections, small pox, etc.). In some non-limiting embodiment or aspects, sensor beacons 102 transmit information associated with a user to beacon bridge 104. For example, sensor beacon 102 transmits temperature or infection information via a one way connectionless protocol, such as communication of information associated with a user to beacon bridge 104 via a connectionless Bluetooth signal. By configuring beacon bridge 104 for WiFi connectivity via gateway 106 (e.g., Wifi piggy backing, etc.), sensor beacon 102 communicates information indirectly to cloud monitoring server 108, such that beacon bridge 104 operating on WiFi or another power intensive protocol eliminates a need for high power connection between sensor beacons 102 over WiFi or another protocol.

In some non-limiting embodiments or aspects, sensor beacon 102 includes a controller (e.g., main processor that binds the sensor with the beacon, etc.). Sensor beacon 102 takes or measures a temperature based on a command from the controller to the sensor to take a number (e.g., a predefined number, a burst, etc.) including many temperatures during this time. Controller can curate the best number out of temperatures, then transmit that number to the beacon portion of the controller. Within the band the sensing side may obtain the body temperature and environmental temperature. Such measured temperatures are packaged and advertised. As soon as in range of one of sensor bridges 104 (e.g., in a threshold proximity, close enough, etc.), the advertisement is picked up. Cloud monitoring server 108 performs the aggregating and data optimization.

In some non-limiting embodiments or aspects, sensor beacon 102 includes a tapping interface to wake up the band, for example, by receiving a specified number of taps to recalibrate or measure the user's temperature.

In some non-limiting embodiments or aspects, sensor beacon 102 comprises a wearable apparatus or provides a shape or capabilities or another wearable apparatus, such as, for example, a wristwatch, a bracelet, etc., and is provided with a first electrode, a second electrode and a third electrode for infection detection. In some examples, sensor beacon 102 includes a first electrode that is provided on a back of a body of the wearable device. In some examples, the second electrode is provided on a surface of a band of the wearable device, which is configured to be in contact with a skin area. In some examples, a third electrode may be an internal sensor capable of measuring another aspect, such as an ambient temperature.

In some non-limiting embodiments or aspects, beacon bridge 104 may be implemented in software, hardware, or a combination of software and hardware to receive and transmit messages (e.g., advertised information, etc.) via connected gateway 106 (e.g., integrated gateway, attached gateway, etc.). For example, connected gateway 106 may have a predefined connection with beacon bridge 104, or alternatively, an integrated connection, whereby information may be communicated. In addition, connected gateway 106 may have a predefined path for communicating information with cloud monitoring server 108 (e.g., via a predefined multi-direction path using a wireless access protocol, etc.).

In some non-limiting embodiments or aspects, sensor beacon 102 waits or sleeps without operating or activating any functionality. In some non-limiting embodiments or aspects, sensor beacon 102 only advertises when the band needs to advertise, such as a predetermined time, a particular mode, or a particular detected condition or aspect of an infectious user. Thus, sensor beacon 102, beacon bridge 104, connected gateway 106, and cloud monitoring server 108 are not connected for the bulk of the time, and are not always sending signals (e.g., are transmitting as needed or required by conditions presented, etc.), and are not maintaining a paired connection (e.g., utilize a connectionless protocol, etc.).

In some non-limiting embodiments or aspects, multiple bands (e.g., sensor beacon devices, etc.) connect to any number of bridges (e.g., gateways) which connect to cloud monitoring server 108. In some examples, one bridge may be programmed or configured for acting as an aggregator, but all bridges are designed to work independently from each other and have no need for a central gateway.

By eliminating a need for a central gateway, the temperature and infection monitoring system 100 can reduce complexity and save power by having all beacon bridges pass the band information directly to the cloud where information is processed (e.g., de-duplicating multiple or redundant information, messages, advertisements, etc.) there, thereby reducing band requirements to pair/connect, and reduce bridge power/complexity requirements by blindly sending most data to the cloud to be intelligently processed.

In some non-limiting embodiments or aspects, cloud monitoring server 108 may provide an association (e.g., assignment, etc.) of a wearable device to a user device. In addition, information or data is sent to cloud monitoring server 108, such as body temperature and environmental temperature to compensate for environmental factors. Temperature data are sent to the cloud to determine the best reading. In this way, terminating devices (e.g., the beginning, first device, etc.) are more efficient and provide less computation of data because computationally intensive operations are effectively transferred to cloud monitoring server 108, which has a much more robust set of access protocols, processing, and storage capabilities for handling such operations. For example, cloud monitoring server 108 provides aggregating and data optimization.

In some non-limiting embodiments or aspects, an anonymous mode may be applied, bands (e.g., sensor beacons, etc.) are not associated to specific users, but instead assigned to monitor devices (e.g., sensor beacons, mobile devices, etc.) and when an event (e.g. high-temperature) is detected in the environment, an agent can determine which monitoring device is advertising the event by either detecting using a receiver device (e.g., a monitoring device receiving messages from the cloud monitoring server, etc.), an application (e.g., an application on a computing device, on a mobile phone, etc.), and/or the like. Additionally, the band itself may display via LED pattern that it is advertising an event. An example would be a day-care setting where children simply pick a random band in the morning and if they start a fever, their LED will blink and the day care personnel can either visually detect or use the mobile application to find the child. This significantly reduces a period of time required to get all children monitored by not having to associate bands to the children.

Cloud monitoring server 108 can determine and provide the optimal or best reading and environmental factor and fine tune to compensate for core temperature and wrist temperature. For example, cloud monitoring server 108 generates a trending indicator set including not just one temperature but a trend of temperatures. In this way, sensor beacon 102 communicates with beacon bridge 104 while users are performing activities in an environment (e.g., a patient care facility, an office, an industrial complex, etc.) such as walking, moving, roaming, standing still, in meetings, or on rounds, while data is sent to beacon bridge 104 and then picked up by gateway 106 and transferred to the cloud.

Mobile device 110 is connected to cloud monitoring server 108 and receives population management data and/or personal management data. Mobile device 110 may include a mobile application for managing and viewing temperature and infection data. For example, mobile device 110 displays trending temperatures in both a micro and a macro view associated with a user or an organization using the application for monitoring individuals in an environment to determine whether the individuals are at risk based on use of temperature and infection monitoring system 100.

In some non-limiting embodiments or aspects, communication network 112 includes one or more wired and/or wireless networks. For example, communication network 112 includes a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

The components of temperature and infection monitoring system 100 may communicate with each other via, for example, two communication networks of the same type as communication network 112. Networks may each be, for example, an internal wired network, a Virtual Private Network (VPN) or the Internet. Networks connecting components of temperature and infection monitoring system 100 may be the same or different networks. In alternative embodiments, all of the components of temperature and infection monitoring system 100 may communicate using a single network (not shown), such as the Internet with the exception of a Bluetooth protocol used between sensor beacon 102 and beacon bridge 104 that use Bluetooth. In yet other alternative embodiments, sensor beacon 102 may utilize more than two protocols (not shown). For example, communication network 112 may be replaced by a first communication protocol for communication between sensor beacon 102 and beacon bridge 104, and a second network and/or second protocol for communication between the other components. In this example, sensor beacon 102 may not be connected with the second network, and beacon bridge 104 may bridge sensor beacon 102 to the second network.

The number and arrangement of systems, devices, and networks shown in FIG. 1 are provided as an example. There can be additional systems, devices and/or networks, fewer systems, devices, and/or networks, different systems, devices, and/or networks, or differently arranged systems, devices, and/or networks than those shown in FIG. 1. Furthermore, two or more systems or devices shown in FIG. 1 can be implemented within a single system or a single device, or a single system or a single device shown in FIG. 1 can be implemented as multiple, distributed systems or devices. Additionally or alternatively, a set of systems or a set of devices (e.g., one or more systems, one or more devices) of temperature and infection monitoring system 100 can perform one or more functions described as being performed by another set of systems or another set of devices of temperature and infection monitoring system 100.

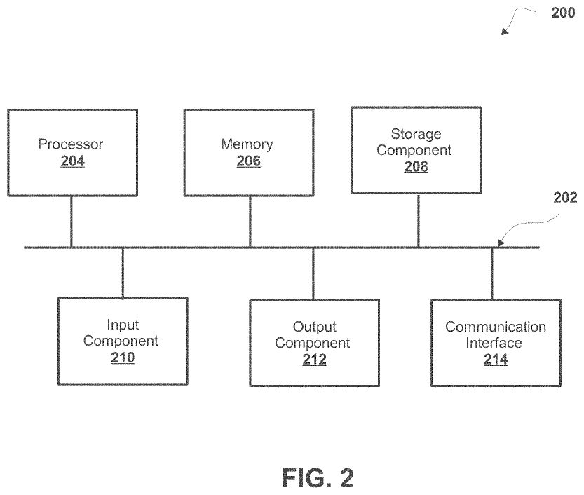

Referring now to FIG. 2, FIG. 2 is a diagram of example components of a device 200. Device 200 can correspond to one or more devices of temperature and infection monitoring system 100, one or more devices of sensor beacon 102, one or more devices of (e.g., one or more devices of a system of) beacon bridge 104, one or more devices of (e.g., one or more devices of a system of) gateway 106, and/or one or more devices of cloud monitoring server 108. In some non-limiting embodiments or aspects, one or more devices of temperature and infection monitoring system 100, one or more devices of sensor beacon 102, one or more devices of (e.g., one or more devices of a system of) one or more sensor beacons 104, one or more devices of (e.g., one or more devices of a system of) gateway 106, and/or one or more devices of cloud monitoring server 108 can include at least one device 200 and/or at least one component of device 200. As shown in FIG. 2, device 200 includes bus 202, processor 204, memory 206, storage component 208, input component 210, output component 212, and communication interface 214.

Bus 202 includes a component that permits communication among the components of device 200. In some non-limiting embodiments or aspects, processor 204 is implemented in hardware, firmware, software, a combination, and/or the like. For example, processor 204 includes a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 includes a random access memory (RAM), a read only memory (ROM), and/or another type of dynamic or static storage device (e.g., flash memory, magnetic memory, optical memory, etc.) that stores information and/or instructions for use by processor 204.

Storage component 208 stores information and/or software related to the operation and use of device 200. For example, storage component 208 includes a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 includes a component that permits device 200 to receive information, such as via user input (e.g., a touch screen display, a keyboard, a keypad, a mouse, a button, a switch, a microphone, etc.). Additionally or alternatively, input component 210 includes a sensor for sensing information (e.g., a global positioning system (GPS) component, an accelerometer, a gyroscope, an actuator, etc.). Output component 212 includes a component that provides output information from device 200 (e.g., a display, a speaker, one or more light-emitting diodes (LEDs), etc.).

Communication interface 214 includes a transceiver-like component (e.g., a transceiver, a separate receiver and transmitter, etc.) that enables device 200 to communicate with other devices, such as via a wired connection, a wireless connection, or a combination of wired and wireless connections. Communication interface 214 can permit device 200 to receive information from another device and/or provide information to another device. For example, communication interface 214 includes an Ethernet interface, an optical interface, a coaxial interface, an infrared interface, a radio frequency (RF) interface, a universal serial bus (USB) interface, a Wi-Fi interface, a cellular network interface, and/or the like.

Device 200 can perform one or more processes described herein. Device 200 can perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions can be read into memory 206 and/or storage component 208 from another computer-readable medium or from another device via communication interface 214. When executed, software instructions stored in memory 206 and/or storage component 208 cause processor 204 to perform one or more processes described herein. Additionally or alternatively, hardwired circuitry can be used in place of or in combination with software instructions to perform one or more processes described herein. Thus, embodiments described herein are not limited to any specific combination of hardware circuitry and software.

Memory 206 and/or storage component 208 may include data storage or one or more data structures (e.g., a database, etc.). Device 200 may be capable of receiving information from, storing information in, communicating information to, or searching information stored in the data storage or one or more data structures in memory 206 and/or storage component 208. In some non-limiting embodiments or aspects, the information may include data (e.g., user information, temperature data, one or more prior temperatures, infection information, temperature trending information, etc.) associated with one or more regions and/or one or more temperature advertisements.

The number and arrangement of components shown in FIG. 2 are provided as an example. In some non-limiting embodiments or aspects, device 200 includes additional components, fewer components, different components, or differently arranged components than those shown in FIG. 2. Additionally or alternatively, a set of components (e.g., one or more components) of device 200 can perform one or more functions described as being performed by another set of components of device 200.

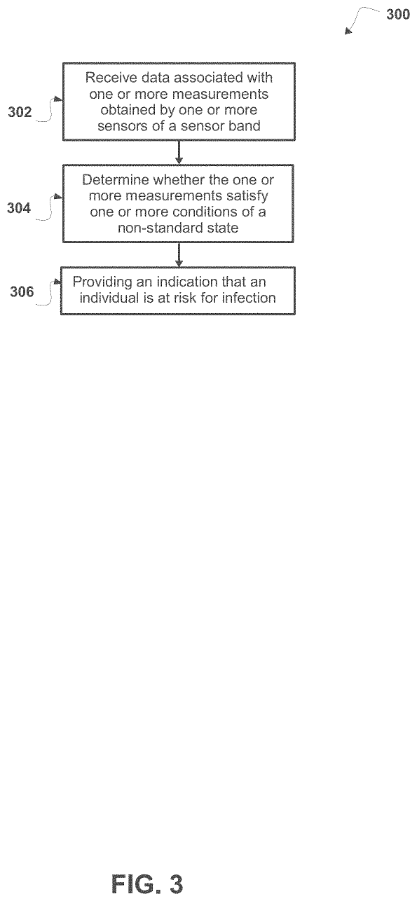

Referring now to FIG. 3, FIG. 3 is a flowchart of a non-limiting embodiment or aspect of a process 300 for generating a site selection. In some non-limiting embodiments or aspects, one or more of the steps of process 300 are performed (e.g., completely, partially, etc.) by temperature and infection monitoring system 100.

As shown in FIG. 3, at step 302, process 300 includes receiving data associated with one or more measurements obtained by one or more sensors of a sensor band. For example, sensor beacon 102 transmits the data associated with the one or more measurements obtained by the one or more sensors by advertising the data associated with the one or more measurements to at least one gateway device.

In some non-limiting embodiments or aspects, instead of connecting to beacon bridge 104, one or more sensor beacons 102 can connect to a mobile phone. For example, in alternate embodiments or aspects, a mobile application connects to sensor beacon 102 and may bridge to cloud monitoring server 108 for early detection and population management. This provides efficiency when using a bridge in industrial applications. Alternatively, a mobile device provides an application in the foreground. In some examples, a modular application API running on a mobile device can operate in the foreground, such that a mobile device is acting as a scanner and a gateway (e.g., bridge, etc.).

One or more sensor beacons 102 (e.g., band 502, band 402, sensor bands, etc.) include a beacon (e.g., advertising beacon, etc.), a sensor (e.g., thermometer, etc.), and a tapping mechanism. Such sensor beacons 102 include bands that can bridge by communicating through beacon bridges. In some examples, sensor beacons 102 are provided to detect and determine, through a sensor, whether a triggering operation satisfying a preset condition is received. In some non-limiting embodiments or aspects, sensor beacons 102 (e.g., beacon capable devices, etc.) include a new framework based on the capabilities of the devices to solve such problems.

In some non-limiting embodiments or aspects, an amount of data transmitted by sensor beacon 102 is minimized. For example, pairing can be adjusted, where certain user groups may not need to pair to the base station. No connecting is used to make a connection for a non-paired connection and sensor beacons 102 (e.g., bands) can communicate using built in beacons to send connectionless messages to beacon bridge 104.

In some examples, beacons are small, wireless transmitters that use low-energy Bluetooth technology to send signals to other smart devices nearby. Beacons connect and transmit information to smart devices making location-based searching and interaction easier and more accurate. However, the beacon and mobile devices are not paired.

In some non-limiting embodiments or aspects, sensor beacon 102 comprises a wearable apparatus comprising: a determining circuit, a detecting circuit and a switching circuit, and an adjusting circuit; wherein, the detecting circuit is configured to detect and determine, through a sensor, whether a triggering operation satisfying a preset condition is received. In some examples, the switching circuit is used for switching the wearable apparatus (e.g., sensor beacon 102) to a preset operating mode when the detecting circuit detects the triggering operation (e.g., a measurement, a proximity to another user, a detectable trend, etc.) satisfying the preset condition, wherein the preset condition is set in advance based on an infectious disease threatening or known to exist in an environment where a user wearing the wearable apparatus may travel, and wherein the preset operating mode comprises a temperature detection mode. In such an example, the determining circuit is configured to determine whether the wearable apparatus is switched to the preset operating mode and the adjusting circuit is configured to adjust mobile device 110 (e.g., to transmit or interact with a mobile application thereon, etc.) to satisfy an operating requirement of a current operating mode when the determining circuit determines that the wearable apparatus has been switched to the preset operating mode.

Beacon bridge 104 will have compact framework for data consumption levels. To maximize battery life and minimize power consumption, control is made of the client and the software running on the bridge to adjust the beacon frequency requirement lower (max 750 milliseconds), and to stretch out or maximize battery performance.

Beacon bridge 104, in some embodiments, may reduce battery charging time by minimization in the number and frequency of messages, eliminating unnecessary monitoring of messages so that beacon bridge 104 is not required to monitor all the time.

In some non-limiting embodiments or aspects, power consumption is adjusted by controlling battery size, advertising frequency, and power output to determine the range (e.g. how far a message can be transmitted, etc.). In addition, the amount of data can be controlled to adjust the power consumption.

Sensor beacon 102 can include an accelerometer to control transmissions. For example, sensor beacon 102 may only transmit when moving or other types of movements to limit the number of messages.

Sensor beacon 102 pushes messages to the bridge device (e.g., beacon bridge 104, etc.). The beacon is designed to not use a connection and can blast the beacon without pairing.

In some non-limiting embodiments or aspects, a beacon sleeps while it is not advertising and can save power and battery life. The beacon may use the accelerometer to advertise only when within range of the bridge.

In some non-limiting embodiments or aspects, power saving is performed by turning the device off if the device is not warm. In such an example, based on a temperature, sensor beacon 102 determines it is on a user, and it is configured to then begin to advertise health information to beacon bridge 104 which is proximate to the user. In some examples, sensor beacon 102 may advertise for only a predefined time, for example, an interval that is preconfigured, and may be automatically defined based on human factors (e.g., previous temperature readings, age, number of contacts, location, etc.).

In some non-limiting embodiments or aspects, an advertising interval is operable to advertise for a period and then sleep. Very frequent advertising can use a great amount of power, by configuring sensor beacon 102, the advertising is limited to reduce the frequency of messages and thereby to reduce power consumption.

In some non-limiting embodiments or aspects, sensor beacon 102 advertises for a predetermined period, such as every 10 seconds. For example, sensor beacon 102 only advertises when it needs to advertise. The temperature is read only when it needs to be taken.

In some non-limiting embodiments or aspects, the thermometer can be activated by tapping the device to get temperature on demand. Tapping can be done whenever, such as when returning to an environment or when entering a building. As long as the band is being warn, the temperature readings are checked.

Sensor beacon 102, and alternatively beacon bridge 104 may generate and provide time stamp, other metrics, attributions to the data, and strip down things that are not needed.

In some non-limiting embodiments or aspects, security is provided based on encryption protocols (e.g., 128 bit encryption, 256 bit encryption, etc.). To fake data, hackers would have to catch media access control (MAC) ID, unique identification (UID), or other device identifiers to fake data. In addition, in order to fake a user, a hacker would need to be physically at the site of sensor beacon 102. In some examples, advertising messages are secured by randomizing the data packet that is being sent and then decoded when it gets to the gateway or cloud to secure data and/or personal identifying information (PII). In addition, PII is secured by storing and associating such data only in the cloud (e.g., name and location). An additional benefit is the decrease in passing identifying information thereby further limiting the amount of bandwidth needed to propagate messages.

If the device is not communicating to the cloud (e.g., not wearing, or not communicating, etc.) the cloud will determine immediately and determine if a connection can be made.

In some non-limiting embodiments or aspects, sensor beacon 102 transmits the data associated with the one or more measurements obtained by the one or more sensors of sensor beacon 102 (e.g., sensor band, etc.) by advertising the data associated with the one or more measurements to at least one gateway device. For example, sensor beacon 102 transmits the data associated with the one or more measurements obtained by the one or more sensors of the sensor band by advertising the data associated with the one or more measurements from a Bluetooth low energy peripheral device included in the sensor band to the at least one gateway device.

In some non-limiting embodiments or aspects, beacon bridge 104 or gateway 106 receives the data associated with the one or more measurements obtained by the one or more sensors of sensor beacon 102. In such an example, beacon bridge 104 forwards the data to cloud monitoring server 108. For example, beacon bridge 104 receives data associated with a temperature measurement of the individual via a Bluetooth protocol from sensor beacon 102 and transmits data associated with a temperature measurement of an environment in which the individual is located to gateway 106. In some examples, sensor beacon 102 transmits using a connectionless Bluetooth protocol. Beacon bridge 104 transmits using a WiFi protocol predefined to connect to cloud monitoring system 108.

In some non-limiting embodiments or aspects, a dataset is provided as compact as possible, and data is transmitted or received one by one. In such examples, gateway 106 or cloud monitoring server 108 places the data in a queue, for example, to store and forward. In some examples, sorting and filtering is provided to the data. For example to block out phony devices or extraneous data or devices. A timestamp may be included, but one way transmission eliminates delays from gateways or other.

As shown in FIG. 3, at step 304, process 300 includes determining whether the one or more measurements obtained by the one or more sensors of sensor beacon 102 satisfies one or more conditions of a non-standard state, wherein the non-standard state is associated with an individual at risk for infection. For example, cloud monitoring server 108 determines whether the one or more measurements obtained by the one or more sensors of sensor beacon 102 satisfies one or more conditions of a non-standard state (e.g., an infection, a specified temperature transition, etc.).

In some non-limiting embodiments or aspects, a terminal (e.g., a handheld device, a mobile device, a computing device, an online computing device, etc.) is provided. For example, the terminal comprises a memory, and a processor, wherein the processor is configured to execute program instructions in the memory, the program instructions, when read by the processor, perform the method described herein. For example, the terminal obtains data and determines whether the one or more measurements obtained by the one or more sensors of sensor beacon 102 satisfies one or more conditions of a non-standard state (e.g., an infection, a specified temperature transition, etc.).

In some non-limiting embodiments or aspects, sensor beacon 102 is configured to switch the wearable apparatus to a preset operating mode (e.g., sending an advertisement alert, etc.) when the detecting circuit detects the triggering operation satisfying the preset condition (determining whether the one or more measurements obtained by the one or more sensors of sensor beacon 102 satisfies one or more conditions of a non-standard state). In some examples, the preset condition is set in advance based on an environment or habits of a user wearing the wearable apparatus. In some examples, the preset operating mode comprises a temperature detection mode. Such that a process of determining whether the wearable apparatus has been switched to a preset operating mode, includes adjusting the wearable apparatus to satisfy an operating requirement of a current operating mode when it is determined that the wearable apparatus has been switched to the preset operating mode.

In some non-limiting embodiments or aspects, cloud monitoring server 108 determines a core temperature of the individual based on the temperature measurement of the individual and the temperature measurement of the environment in which the individual is located. In some examples, cloud monitoring server 108 obtains one or more measurements from a plurality of sensor beacons via a beacon bridge, wherein the measurements are performed on a plurality of users of a plurality of sensors, and the measurements for each of the plurality of users satisfies a specified condition of a non-standard state (e.g., an infection, a specified temperature transition, etc.). In such an example, when the measurements for each of the plurality of users satisfies a specified condition of a non-standard state (e.g., an infection, a specified temperature transition, etc.), a message with detectable data is transmitted to a mobile application for generating and displaying a message in a mobile application.

In some non-limiting embodiments or aspects, cloud monitoring server 108 determines or compares the core temperature of the individual to a predetermined range of core temperatures corresponding to a high-temperature condition included in the one or more conditions non-standard state.

In some non-limiting embodiments or aspects, cloud monitoring server 108 determines that the core temperature of the individual satisfies a predetermined range of core temperatures corresponding to the high-temperature condition included in the one or more conditions of the non-standard state.

In some non-limiting embodiments or aspects, cloud monitoring server 108 determines that the one or more measurements obtained by the one or more sensors of sensor beacon 102 represents one or more trends.

In some non-limiting embodiments or aspects, normalization of temperature includes a burst (e.g., taking many readings and getting the best pick of readings, etc.). The beacon transmits the optimal or best pick for the body temperature and environmental temperature. The cloud can get the sensor reading and environmental factor and fine tune to compensate for core temperature and wrist temperature.

In some non-limiting embodiments or aspects, a cloud core function set includes not just one temperature but a trend of temperatures.

In some non-limiting embodiments or aspects, cloud monitoring server 108 determines whether the one or more trends indicate that the individual is at risk for infection. For example, cloud monitoring server 108 determines that the one or more measurements obtained by the one or more sensors of sensor beacon 102 represents one or more trends. In some non-limiting embodiments or aspects, cloud monitoring server 108 determines or compares the one or more trends to the profile corresponding to the individual.

In some non-limiting embodiments or aspects, cloud monitoring server 108 determines whether the one or more trends indicate that the individual is at risk for infection based on comparing the one or more trends to the profile corresponding to the individual, such that when providing the indication that the individual associated with sensor beacon 102 is at risk for infection an indication is provided. Cloud monitoring server 108 provides the indication that the individual associated with sensor beacon 102 is at risk for infection based on determining that the one or more trends indicate that the individual is at risk for infection based on comparing the one or more trends to the profile corresponding to the individual.

As shown in FIG. 3, at step 306, process 300 includes cloud monitoring server 108 providing an indication that an individual associated with sensor beacon 102 is at risk for infection.

In some non-limiting embodiments or aspects, cloud monitoring server 108 determines and mobile device 110 provides the indication (e.g., in a user interface, in an application, etc.) that the individual is at risk for infection based on determining that the core temperature of the individual satisfies the predetermined range of core temperatures corresponding to the high-temperature condition included in the one or more conditions of the non-standard state.

In some non-limiting embodiments or aspects, cloud monitoring server 108 determines a core reading based on temperature readings and take environmental readings into consideration. Such core readings are used to generate a user interface, such as, user panels to display a temperature accumulation showing or highlighting a temperature trend using a history of measurements to show how a user is doing or an infection.

With trending factor, cloud monitoring server 108 can adjust (e.g., eliminate anomalies, false positives, false negatives, fluke, etc.) and can make better judgment based on further monitoring, come back or return to take temperature, take a temperature directly of person, and/or the like. An entire office or facility may be using a monitoring system. Machine learning can be used on the data to learn where the hotspot started, and additional other dimensions, such as time, location, and/or the like may be added to the data to provide further aspects or embodiments.

In some non-limiting embodiments or aspects, cloud monitoring server 108 receives temperature readings over a period of time (e.g., 48 hours, 1 week, etc.) and generates an ongoing visual representation of how a user is doing. A threshold temperature is used to represent or determine the levels. The display of a trend can be provided to represent the trend in the user interface prominently, or alternatively to present the trend as an information bundle which a user may easily comprehend.

The user interface provides efficiencies and a technical improvement between the displayed interface and the underlying system to provide continuous temperature monitoring and save battery and bandwidth, provide efficiencies when using historical data or computing information based on such historical data.

In some non-limiting embodiments or aspects, sensor beacon 102 transmits temperature or other user information to the cloud for each predetermined period (e.g., 5-10 microsecond, 2 minutes, 20 minutes, etc.). Trending bars are provided in a user application (e.g., user interface, mobile application, cloud application, etc.) which include units that represent one hour. If three readings per hour, unit is going to be average of that to represent an hour, such that the period and units can be adjusted to control the resolution of the trending bar. Display a trend, to eliminate data for example, if someone runs outside or runs to the bathroom, their temperature may go up, but averaging over the hour serves to eliminate spikes that may be caused by external situations. User interface shows the last reading as a ghost figure which indicates it is about to be set, but it is not yet set.

In some non-limiting embodiments or aspects, colors correspond to the temperature readings. For example, the colors red (e.g., warning, danger, etc.), yellow (e.g., caution, suspect, etc.), blue (e.g., OK, etc.), and gray (e.g., offline, no-data, etc.) represent temperature thresholds or ranges. Height of the units may be used to indicate a character of the data, such as the amount of data used to determine the unit.

In some non-limiting embodiments or aspects, the last bar is the latest reading representing the last hour, showing the latest reading and the average of the hour up to that time. A temperature onset pattern can be predicted by checking multiple times. In hospitals, a sudden rise from where a patient was before, and a sustaining of the rise over a period of time.

In some non-limiting embodiments or aspects, cloud monitoring server 108 receives data associated with registration of the one or more sensors from sensor beacon 102 via beacon bridge 104. Cloud monitoring server 108 registers, associates, authenticates sensor beacon 102 with the individual. In such a case, cloud monitoring server 108 generates a profile corresponding to the individual based on the data associated with the one or more measurements obtained by one or more sensors of sensor beacon 102.

In some non-limiting embodiments or aspects, when providing the indication that the individual associated with sensor beacon 102 is at risk for infection, cloud monitoring server 108 may provide the indication that the individual associated with sensor beacon 102 is at risk for infection based on determining that the trend indicates that the individual is at risk for infection.

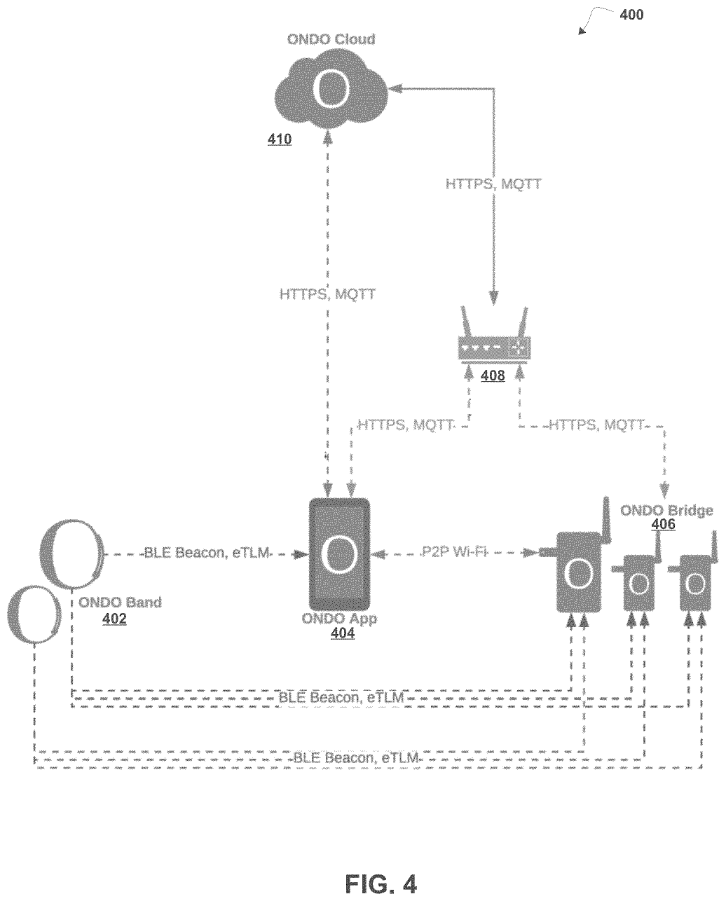

Referring now to FIG. 4, FIG. 4 is a diagram of an example temperature and infection monitoring system 400 for monitoring individuals in an environment to determine whether the individuals are at risk in which devices, systems, methods, and/or products described herein, may be implemented. As shown in FIG. 4, temperature and infection monitoring system 400 includes band 402 (e.g., one or more sensor bands 402, etc.), mobile application 404, bridge 406, gateway 408, and cloud server 410 (e.g., one or more cloud servers 410, one or more cloud computers, etc.).

Systems and/or devices of temperature and infection monitoring system 400 can interconnect as shown in FIG. 4, via wired connections, wireless connections, or a combination of wired and wireless connections for early detection and management of infectious diseases. In some non-limiting embodiment or aspects, band 402 transmits via a bluetooth beacon. For example, bluetooth beacons can be configured to perform as hardware transmitters which provide low energy (LE) broadcasts along with their identifier to nearby portable electronic devices. In this way, band 402 transmits, in some non-limiting embodiments or aspects, to mobile application 404, bridge 406, or other devices of the system. For example, efficiency is gained as mobile application 404, bridge 406, or other devices are programmed to perform actions when in close proximity to a beacon of band 402. Likewise, mobile application 404, bridge 406, or other devices may also transmit information to band 402 using the same beacon, when in range.

In some non-limiting embodiments or aspects, an identifier and several bytes representing the band information are sent with it and can be used to determine the device's physical location, track users, or trigger a location-based action on the device such as a check-in on cloud server 410. In some examples, band 402 can be configured for distributing messages via only a bridge within a range of band 402, thereby decreasing collisions that may occur when a message is transmitted through numerous paths. Thus, when a user is moving around in an office space, moving from a room to another room, can be configured to send through only a single point. This provides an indoor positioning system, which aids mobile application 404 and bridge 406, as band 402 only transmits to the devices in close proximity. This may also provide location information for determining contact tracing using triangulation techniques to determine a user's approximate location or contacts during a period of time.

In some non-limiting embodiments or aspects, band 402 may provide only a one-way transmitter to receiving mobile application 404 or bridge 406, such that mobile application is installed on the mobile device to interact with the beacons. This ensures that only mobile application 404 can track users, without intruding on the user to approve transmission, as they passively walk around in the office space.

Band 402 transmits information associated with a user to bridge 406. For example, band 402 transmits temperature or infection information via a one way connectionless protocol, such as communication of information associated with a user to bridge 406 via a connectionless Bluetooth signal. By configuring bridge 406 for WiFi connectivity via mobile application 404 or gateway 408 (e.g., https, MQTT, WiFi piggy backing, etc.), band 402 communicates information indirectly to cloud server 410, such that bridge 406 operating on WiFi or another power intensive protocol increases efficiency by eliminating a need for high power connections between band 402 to mobile application 404 or cloud server 410 over WiFi or another protocol.

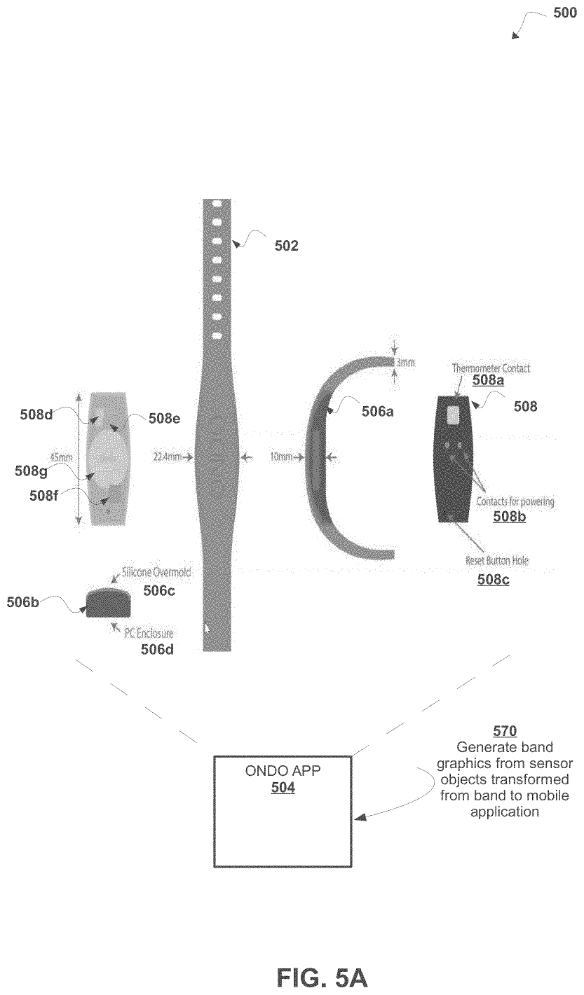

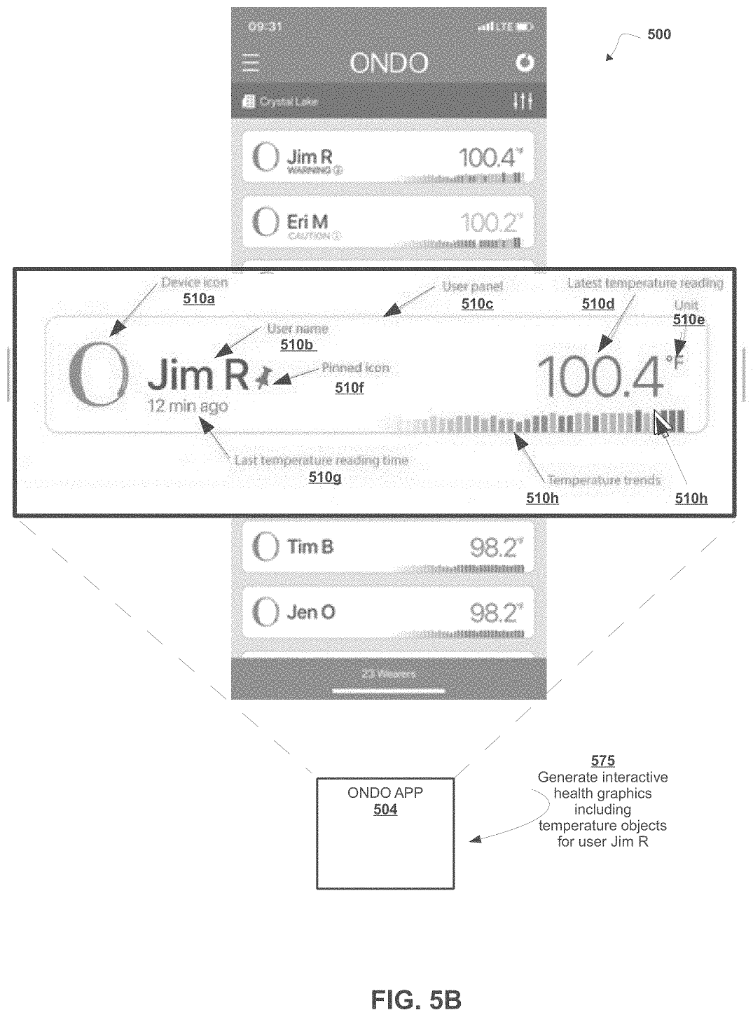

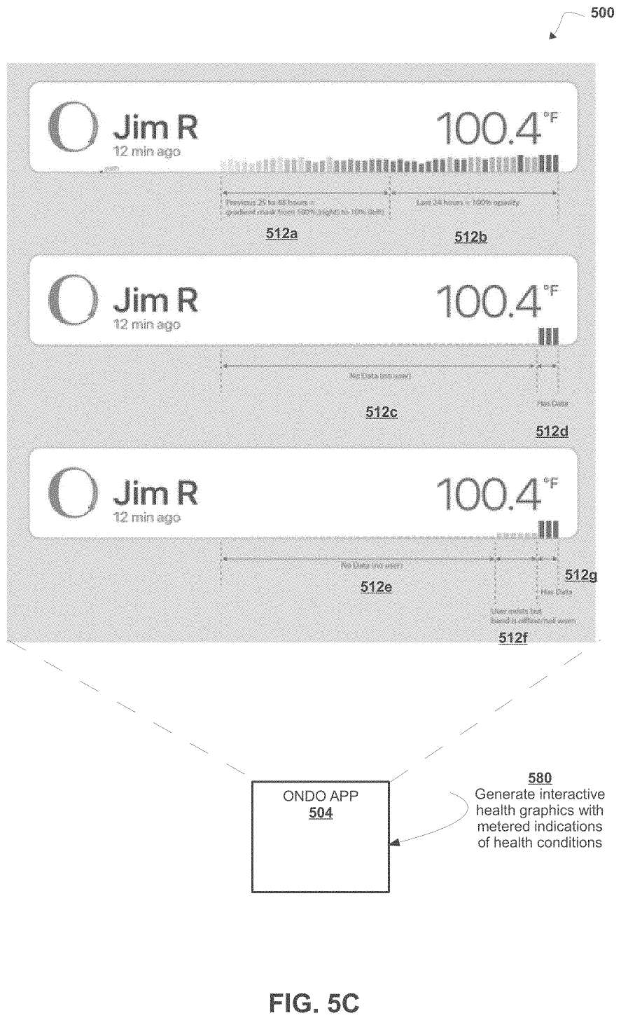

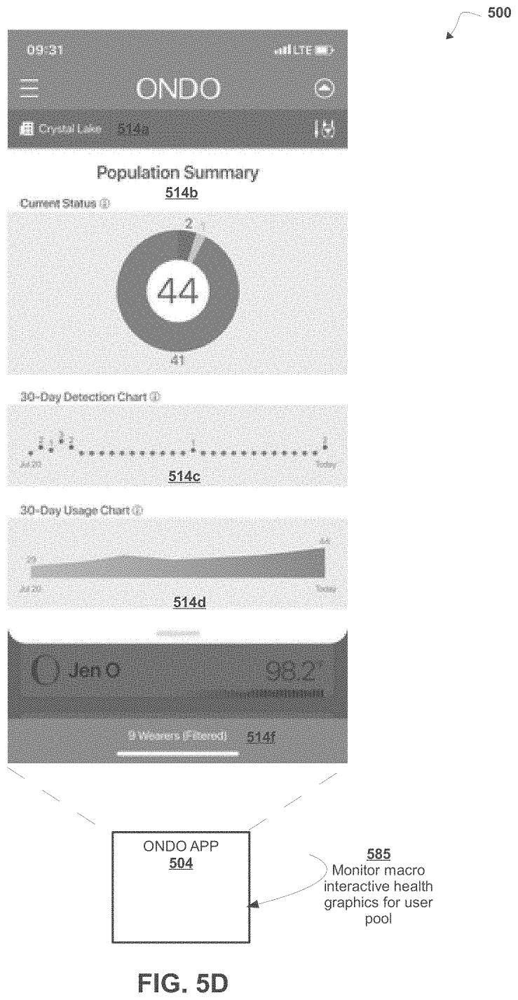

Referring now to FIGS. 5A-5D, FIGS. 5A-5D are illustrations of an overview of a non-limiting embodiment of an implementation 500 relating to one or more processes disclosed herein. In some non-limiting embodiments or aspects, one or more of the steps of implementation 500 are performed (e.g., completely, partially, etc.) by one or more components or processes of temperature and infection monitoring system 400 including a sensor, such as band 402 (e.g., one or more sensor bands 402, one or more sensor processors, one or more sensors, a stack of sensors, etc.), mobile application 404 (one or more displays of mobile application 404, one or more mobile applications 404, one or more functionalities of mobile application 404, etc.), bridge 406 (one or more connections of bridge 406, one or more transmissions of bridge 406 one or more health messages of bridge 406, etc.), gateway 408 (one or more processors of gateway 408, one or more routings of gateway 408, etc.), cloud server 410 (e.g., one or more cloud servers 410, one or more cloud computers, one or more cloud processors, etc.), and a health monitor data source (e.g., one or more computing devices operating gateway 106, etc.). In some non-limiting embodiments or aspects, one or more of the steps of implementation 800 are performed (e.g., completely, partially, etc.) by another device or a group of devices separate from or including temperature and infection monitoring system 400, such as one or more device of (e.g., one or more devices of a system of) a cloud database, a 3^{rd} party protected health information (PHI) server, etc.).

As shown by reference number 570 in FIG. 5A, mobile application 504 generates band graphics from sensor objects transformed from band to mobile application. For example, mobile application 504 obtains and provides graphical objects describing or portraying a user in a detectable manner, associated with and/or using a sensor band which captures sensor data and transmits a message which is transformed (e.g., in a cloud server 410) into graphics indicating temperature, infection, health parameters, or frequency occurrences.

In some non-limiting embodiments or aspects, band 502 generates sensor objects. The band is designed with a flexible silicone coating outside a more firm surface. Side view 506a of band 502 and cross section 506b of band 502 show the shape and contour of the band, with silicone over mold 506c having a compartment including an enclosure 506d on an underside 508 of band 502.

In such an example, underside 508 of computer 506b comprises devices capable of measuring, transmitting, powering, and activating band 502. For example, thermometer 508a (and contacts), power button and contacts for powering 508b, reset button 508c, band computer 508d, accelerometer 508e, band beacon 508f, and battery 508g are provided in band 502. One of ordinary skill would understand that more than one device may share a capability. The band computer 508d is configured to process the data collected by the sensors (e.g., thermometer 508a, accelerometer 508e, band beacon 508f, etc.), control the operations of the band 502 and the sensors, and process the collected infection detection data. In some non-limiting embodiments or aspects, program instructions are stored in memory (e.g., band memory that is on the band 502, and accessible by the components of the band 502, such as computer 508d, etc.) or are downloaded from the cloud, and are read by the computer 508d to perform the following operations: determining whether the band 502 has been switched to a preset operating mode. Based on the operating mode, computer 508d displays infection detection data.

In some non-limiting embodiments or aspects, band 502 includes modes comprising an ID, mode definition, and power consumption (e.g., band computer 508d is programmed to generate modes based on the collected infection detection data, etc.). For example, a first mode may include a storage mode. In some examples, band 502 may be stored or transported in a storage mode (e.g., shipping mode from factory during transportation and storage, etc.). For example, band 502 may not include active sensors or active beacons. In some examples, a mechanical switch, such as reset button 508c is activated to change the mode out of storage mode.

In some non-limiting embodiments or aspects, band 502 includes an active mode (e.g., the standard operation mode, etc.), and the bands are expected to stay in this mode (e.g., the majority of active life, 24 hours/7 days a week). In active mode, the sensors run per regular interval and the beacon transmits on a regular interval. Band 502 may include a mechanical switch, such as reset button 508c that is activated to change the mode (e.g., 6 s to 9 m 59 s to enter into storage mode, 10 seconds to power cycle, etc.).

In some non-limiting embodiments or aspects, band 502 includes an on-demand reading (ODR) mode, such that band 502 goes into ODR mode by user input, but reverts back to active mode once the ODR mode times out. In ODR mode, sensors of band 502 are immediately activated upon entering the mode, and beacon activity is heightened to a high frequency for predetermined period. In some examples, additional modes include a sleep mode to put band 502 into an at rest state, where all functions are deactivated. In addition, an unworn mode may be used to deactivate some features of band 502, such as periodic automatic temperature readings.

In some non-limiting embodiments or aspects, thermometer contact 508a of band 502 takes measurement of skin temperature at a set interval, and band computer 508d of band 502 passes a reading (e.g., “xx. x” ° C., “xx. x” ° F.) to band beacon 508f for advertisement. This procedure is configured to occur on a periodic interval (e.g., 10 minutes). Band 502 is configured to measure ambient temperature at a set interval (e.g., 10 minutes, etc.), and then pass a reading (e.g., “xx. x” ° C., “xx. x” ° F.) to band beacon 508f for advertisement. In some examples, band 502 is configured to capture readings by accelerometer 508e during temperature reading, categorize the raw reading by range, and pass a single digit category number to band beacon 508f side for advertisement to cloud. For example, readings by accelerometer 508e could be digitized based on a number 0-5, where 0 is reserved for on-demand temperature reading, 1 is the lowest motion level, and 5 is the highest motion level.

In some non-limiting embodiments or aspects, for an ODR in band 502, a mechanical switch under the initial silicon “O” of “ONDO” logotype on band 502 (e.g., an “Ondo Switch” is a hidden button provided for operating specific applications or aspects thereof, such as those that perform an on-demand reading and/or association process between band and wearer, etc.) is activated to trigger on-demand skin and ambient temperature readings (ODR). For example, when ODR is triggered, both non-connectable (NC) and connectable (C) advertisements are advertised continuously for a period of time (e.g., advertisement interval and duration: NC: 5 times, 500 ms apart, then C: 5 times, 500 ms apart, repeat). In some examples, the function is configured to time out: 30 seconds (e.g., goes back to active mode). The beacon set the category number of accelerometer reading to 0 to flag ODR trigger, if the skin temperature reading is above threshold upon an on-demand temperature reading, blink LED for a period of time (e.g., threshold: 38.0° C. for blinking, and ON for 100 ms, OFF for 500 ms, Repeat 1-2 for 5 times). In some examples, band 502 then advertises non-connectable advertisement with sensor data and connectable advertisement for over the air readings (OTA) and configuration at a set interval. (e.g., every 10 minutes). The advertisement interval can be configured to sustain battery 508g.

In some non-limiting embodiments or aspects, band 502 is configured for beacon power output to achieve 25 m of practical range indoor and may be set to TX at a specified frequency (e.g., 4 decibel milliwatts (dBm)). Band 502 is additionally capable of over the air direct messaging with user information, without the user physically interacting with band 502 (e.g. pressing Ondo Switch). In this example, if direct from user session is interrupted or times out (e.g., 60 seconds), go back to operating state. Power cycle reboot is activated by holding the mechanical switch for 10 seconds. When the device is powered up, the LED turns ON for 1000 microseconds and then OFF.

The ONDO switch is held for 6 seconds when the Band is in active or in ODR mode. In some examples, when the button is pressed and held for 6 seconds, the LED blinks ON for 100 microseconds, OFF for 250 microseconds, ON for 100 microseconds, and OFF. The user has to release the button before the holding time hits the 10-second mark to execute this. In some examples, if the user does not release the button, the Band will reboot at the 10-second mark.

In some non-limiting embodiments or aspects, band 502 is configured to detect an unworn state based on thermometer contact 508a obtaining a skin temperature reading threshold (e.g., body temperature reading is below 31.0° C.). In some examples, band 502 is configured to enter sleep state after a set grace period for the beacon advertisement until the accelerometer detects motion.

After the band 502 is in sleep mode state, there may be no interval temperature reading, no advertisements (e.g., after set grace period), or any other activity, until a detectable motion is provided to wake up the band 502. For example, band 502 can be configured to detect a double tap. At that time, a final reading of thermometer contact 508a below a threshold will be advertised for a period of time. In some examples, temperature reading based detection might not be suitable and accelerometer 508e is used instead, with a preconfigured threshold to account for detectable movement.

Upon waking up, band computer 508d rests for a predetermined period (e.g., 60 seconds) before reading the skin and ambient temperatures, unless ODR is triggered. In some examples, band beacon 508f stops advertisement messages when an unworn state is detected after a set grace period (e.g., every 5 minutes).

In some non-limiting embodiments or aspects, power profiling models include an expected typical use case where band 502 is powered on when deployed and stays on thereafter, providing one ODR per week, is worn 24/7, with one detectable threshold fever (e.g., causing the led to blink) by ODR every 6 months, over the air capability not included. In some non-limiting embodiments or aspects, power profiling models include a more intensive use case where band 502 provides default values and behaviors, including transmitting above TX 4 dBM, a power up when deployed such that functions stay on thereafter, seven ODR per week, worn 24/7, and one threshold fever detection (e.g., led blinking) by ODR every month, with over the air capability. In some non-limiting embodiments or aspects, power profiling models include a lighter than typical use case, powers up when deployed and stays on thereafter, with one ODR per month, band 502 worn 12 hours a day, and no over the air capability.

As shown by reference number 575 in FIG. 5B, mobile application 504 generates interactive health graphics including temperature objects for user Jim R. For example, mobile application 504 generates interactive health graphics including temperature objects for user Jim R (e.g., graphics indicating temperature, infection, health parameters, frequency occurrences, etc.).

Mobile application 504 generates or obtains a dashboard that serves as a main point for the application users, having at least two sections (e.g., list view and summary view). The list view obtains wearer data (e.g., a subject user or users) and generates a wearer details panel.

In some non-limiting embodiments or aspects, mobile application 504 generates or obtains a dashboard layout, such as a main screen of the mobile application, generating a detectable view of the population (e.g., a view that is quickly detectable, etc.). For example, mobile application 504 generates or obtains the dashboard shown in FIG. 5B which is made of the following general elements: a title bar, a sub navigation bar, wearer cards.

In some non-limiting embodiments or aspects, mobile application 504 includes a title bar at the very top part of the dashboard screen. The title bar includes at least one of a menu button, a branding logotype (e.g., device icon 510a), and a summery drawer button. In some examples the title bar can present different versions of these items.

In some non-limiting embodiments or aspects, mobile application 504 includes a sub navigation bar that includes at least one of a facility selector button 514a, sort button, and filter button.

In some non-limiting embodiments or aspects, mobile application 504 includes a footer bar. At the bottom of the card list, the footer bar provides a footnote telling how many wearers are shown.

In some non-limiting embodiments or aspects, mobile application 504 generates and displays a wearer card (e.g., user panel 510c). For example, one user panel 510c contains all of a wearer's health data and/or information. A user panel 510c can be obtained and arranged in a list or other arrangement, where each card can be tapped to open a wearer summary panel. In some non-limiting embodiments or aspects, each user panel 510c contains at least the following: a device icon 510a, a username 510b, a user panel 510c, the latest temperature reading 510d, a measurement unit 510e, a pinned icon 510f, the last temperature reading 510g for the identified user (e.g., in ° F. (default) or ° C.), and temperature trends 510h showing the identified user's historical data in a detectable graphic (e.g., 48-hour temperature trends arranged for quickly detectable issues or conditions with the trends as explained below). In some examples, mobile application 504 generates notable state text with “I” icon (not shown if there is no notable state).

Mobile application 504 generates graphic colors for detecting trends and health information generated from user health data. For example, orange is a main color, and used in several components of mobile application 504, including the title bar; white is displayed for most icons and text on dark background; darker orange is displayed in status bar; red color is displayed for warnings, alerts and other priority elements; yellow is displayed when caution should be detectable, such as, for semi-high temperature readings; blue is displayed for “OK” states and other branding elements as complementary color to the orange; gray is displayed for disabled and offline element; dark gray is displayed for most text on light background like last temperature reading time stamp (when it's not over 2 hours); light gray is displayed for dashboard content background; dark brown is displayed for secondary text on dark background like the temperature reading disclaimer at the bottom; and dark yellow is displayed for secondary icons on orange background, for example, the “I” icon located at the bottom (e.g., or some other icon or branding that can serve as a trigger).

Each user panel 510c (e.g., user panel 510c) represents a band wearer. Tapping on the user panel 510c will open the details view of the band wearer. The user panel 510c can be arranged in different orders or filtered based on the wearer.

This UID shows the band wearer's name (e.g., 510b username). Mobile application 504 can set how the name is displayed in the settings. User panel 510c can be configured with notable states that will show up at the top of the list regardless of a sorting criteria set by the user. Within those, priority can be obtained in the matrix below and will be used to prioritize, such that, for example, if within a type of state, the wearer with the latest reading will get the higher spot.

A wearer is in “OK” state (e.g., stable, in a non-notable state, etc.) when wearer's skin temperature is within the OK range (defined separately), and wearer has 48 hours of trending data (e.g., temperature trend 510h). In an example, when a wearer is outside the OK state, mobile application 504 generates a notable state message which indicates the health issue. For example, if there is a notable state with band 502, the notable wearer state will overwrite. In another example, if the notable band state is preventing the detection of a notable wearer state (e.g. a band is offline so a warning temperature can't be alerted), the notable state message of band 502 persists. Mobile application 504 may generate a notable state message that is followed or accompanied by a generation of an “I” icon in mobile application 504, which gives the user additional information in a dialog box when tapped.

In some non-limiting embodiments or aspects, notable band states include “OK” state when a band is OK (e.g., a non-notable state), this can only occur when battery voltage of band 502 is above threshold, band 502 is worn by the wearer (e.g., skin temperature reading is above a lower threshold), and the last data that reached the cloud is no more than 2 hours old. When band 502 is outside the OK state, several detectable indications can be given, band icon changes with a modifier, notable state message indicates the issue unless it's overwritten by notable wearer state, notable state message is followed by “I” icon which gives the user additional information in a dialog box when tapped.

In some non-limiting embodiments or aspects, a warning notifier indicates the latest skin temperature reading is at or above the user defined temperature (per wearer, default number is given for new wearer) threshold, either by number or deviation (e.g., warning in red); caution notifier indicates the latest skin temperature reading is below but within 0.5° C. of the user defined (per wearer, default number is given for new wearer) threshold, either by number or deviation (e.g., CAUTION in caution yellow); and new wearer notifier indicating a wearer is new to the system and does not have sufficient trend history. This indicates the user should be aware the deviation based alert will not work (e.g., NEW WEARER notifier indicating blue at 60% opacity or simulated lighter color at 100% opacity); low battery notifier indicating battery voltage reading is below the defined threshold. The band is still functioning, this state message will be overwritten if there is a notable wear state(e.g., LOW BATTERY in caution yellow); offline bridge/cloud notifier indicating mobile application has not obtained new reading in the last 2 hours, and may include the wearer being out of range, the band being damaged or battery completely depleted (e.g., OFFLINE in gray); and an unworn notifier indicating the latest skin temperature reading is at or below ONDO defined lower threshold (e.g., NOT WORN in offline gray).

As shown by reference number 580 in FIG. 5C, mobile application 504 generates interactive health graphics with metered indications of health conditions. For example, mobile application 504 generates detectable health graphics with multi-dimensional objects unexpanded as detectable metered graphical health conditions.

In some non-limiting embodiments or aspects, mobile application 504 generates or obtains the latest temperature reading 510d based on the last temperature reading (e.g., skin temperature, body temperature, etc.), a reading for the wearer, or alternatively, based on a last temperature reading while in an OK state. In such a case, temperature unit 510e is configured for ° F. or ° C. and is shown after the temperature numbers. ° F. is the default settings, and the admin user can change it in the setting.

In some non-limiting embodiments or aspects, temperature trends are updated based on graphic detectable objects. In some examples, graphic detectable objects show the historical progression of the temperature readings. Each bar represents average reading within an hour and the entire graphic detectable objects can show up to 48 hours of historical data. In an example, at 512b the latest 24 hours are shown at 100% opacity. Also, a gradient mask can be placed on top of 25th to 48th bars as shown at 512a, making the visibility 100% at the 25th hour down to at least 10% at the 48th hour. The far right bar represents the last hour that has not finished, mobile application 504 generates this bar to display the current average for the ongoing hour, and generates each bar to represent from x:00 to x:59 of local time (phone's time).

In some non-limiting embodiments or aspects, colors are used corresponding to the temperature reading colors: warning red, caution yellow, ok blue, offline gray (also used for no-data). Generated bar heights are based on the temperature reading following convention: no-data because no-wearer at 512c and 512e, provides fixed very low height (e.g., 10%, etc.); and offline or not worn shown at 512f, provides fixed low height, but at a steeper height than no-user (e.g., 30%, etc.). In some non-limiting embodiments or aspects, eligible readings 512d and 512g have a height that is greater than a height for a bar corresponding to “not worn” or “offline” readings. For example, eligible temperatures from 35° c. to 40° c. represent 60% of the total height, while anything beyond 40° f. will be 100% of bar height. In some examples, anything below 35° c. will be at 40% bar height (e.g., 40% is the lowest eligible temperature). In this case, any temperature below the lowest eligible temperature is adjusted for height between 40% bar height and maximum height, accordingly, temperature changes and spikes over a time period are efficiently and accurately detectable with just a quick glance.

Mobile application 504 can also generate color thresholds for eligible readings. For example, color can be changed (e.g., to yellow) when the temperature measured is above an even higher threshold. In such an example, color can be changed (e.g., to red) when the temperature measured is above an even higher threshold.

In some non-limiting embodiments or aspects, mobile application 504 generates or obtains the last reading from band 502 (as described above), displaying information generated for the wearer panel, and to supplement detectable trends.

Mobile application 504 can be used to add a wearer and wearer details, and serves as the main hub for adding, changing, deleting, and accessing wearer info, band management and wearer-specific settings. In some examples, a back button takes the user back to the list screen. If there are changes made, the dialog box can confirm a discard of a change. If the user has swapped the band and a change is discarded, an originally associated band is still going to be used.

In some non-limiting embodiments or aspects, a save button saves any changes made to a wearer. In an example where the user has swapped the band and saved the change, the new band is going to be associated with the wearer and the originally associated band that is made available for another wearer.

Wearer information includes, in addition to a band ID, a user name 510b of FIG. 5B (e.g., configured with at least either first name+last name or ID needs to be filled). The band ID for band 502, in some non-limiting embodiments or aspects, comprises only the last 6 digits of a MAC ID, or at least only the last 4-6 digits will be shown in mobile application 504. Band ID can be viewed, changed, or generated by entering (e.g., with a tap) the band details screen.

Mobile application 504 may include wearer settings that are configurable by the user or an administrator of the system. In an example, a wearer group is generated to select from defined groups, an alert threshold can allow a user to set skin temperature threshold down to 1 decimal, and an alert spike allows a user to set skin temperature deviation from baseline down to 1 decimal.

As shown by reference number 585 in FIG. 5D, mobile application 504 monitors macro interactive health graphics for a user pool. For example, mobile application 504 generates interactive health graphics with metered indications to monitor detectable macro health graphics (severity, timeliness, frequency of conditions, duration of condition, etc.) for a user pool (e.g., generating, obtaining, or displaying a single instruction that expands automatically into a set of indicators showing how a contained population performs in relation to a particular health condition, behavior, risk, etc.).

In some examples, mobile application 504 generates interactive health graphics with metered indications comprising a summary view that provides modules to generate or display detectable health data obtained via band 502 or other data sources. The application menu can be accessible from the dashboard title bar icon. A filtering screen accessible from the dashboard title bar icon and secondary screens inside of application menu include wearer management and settings account information (e.g., facility management, user management, bridge management, etc.).

In some non-limiting embodiments or aspects, mobile application 504 includes a summary drawer. The summary drawer shows the at-a-glance summary of the total population within the selected group(s) (e.g., location 514a). A facility selector is linked to the detectable health data that is viewable on the dashboard. If a change is made to the facility data, it will be reflected on the dashboard as well. For example, at the top of the list, there is a facility selector. Facility selector can be administered to change facilities. This is linked to what mobile application 504 generates and displays on the dashboard. Facility will be reflected on the dashboard as well. For facility administration, the interface is fixed and not interactive, just serving as facility name label.

For example, an expanded population summary 514b for location 514a, shows that 44 detectable users, includes 41 detected healthy users, 2 users detected with a confirmed illness, and 1 user detected with symptoms. In one example, mobile application 504 generates or obtains a 30-day detection graphic 514c, showing detected activity dots (or rectangles) and detected numbers of illness. In another example, mobile application 504 generates or detects a 30-day usage 514d for a graphical expansion and detectable trend, the 30-day usage 514d can be limited by user filter 514f, providing viewing of detectable metered graphical health conditions for only specified users.

In some non-limiting embodiments or aspects, mobile application 504 includes a facility selector interactive for corporate roles, not interactive (acts as label) for facility roles. Selector function provides interactive selection and acts as a facility name display. In some non-limiting embodiments or aspects, mobile application 504 sorts and filters, using graphic activation to activate a sort or filter panel. The user can dismiss the panel by tapping the “close” icon, tap on the blank (darkened) area, or drag down the panel by the grabber. In some examples, the actual update of the dashboard can be done dynamically as you change the selections or wait until the user hits close (or dismiss the screen by other methods). For example, an icon of mobile application 504 is updated or overwritten with a “filtered” budge (e.g. a change or alteration that is free form and related to a filter or some other aspect of the detectable information) when a filter is applied. In the current implementation, that would mean at least one group is hidden. When there is at least one wearer in a notable state in those hidden groups, the “!” budge may be presented. When all groups are shown, there will be no budge (e.g., L: not all groups are shown, C: notable state wearer in hidden groups, R: all groups shown).

Wearers in notable state always shows up top. For example, if name based sorting is selected, wearers with only a user ID will show up after those with names. If user ID based sorting is selected, wearers with only name will show up after those with IDs. This selection is local to the app/phone, and not synced across the account.

In some non-limiting embodiments or aspects, mobile application 504 includes filter groups. The user can choose one or more pre-defined groups to filter. In some examples, by default, all groups are selected. This selection is local to the mobile application or can be synced with cloud server 410 and synced across the account. At least one group must be enabled. If a group is selected, the toggle switch should be “stuck on” by showing the enabled color and position, but faded color or an indicator explaining the view. In an example, mobile application 504 must enable a 2nd group before turning off a 1st group (e.g., the 2nd group enabled becomes the only group enabled). If there is a notable state wearer in groups that are hidden, budge “!” is displayed next to that group (either by the name or toggle). The mobile application 504 may generate and display a hint panel based on operation of the budge “!”.

In this description, some functions and operations are described as being performed by or caused by software code to simplify description. However, such expressions are also used to specify that the functions result from execution of the code/instructions by a processor, such as a microprocessor.

Alternatively, or in combination, the functions and operations as described here can be implemented using special purpose circuitry, with or without software instructions, such as using application-specific integrated circuit (ASIC) or field-programmable gate array (FPGA). Embodiments can be implemented using hardwired circuitry without software instructions or in combination with software instructions. Thus, the techniques are limited neither to any specific combination of hardware circuitry and software, nor to any particular source for the instructions executed by the data processing system.

While one embodiment can be implemented in fully functioning computers and computer systems, various embodiments are capable of being distributed as a computing product in a variety of forms and are capable of being applied regardless of the particular type of machine or computer-readable media used to actually effect the distribution.

At least some aspects disclosed can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

Routines executed to implement the embodiments may be implemented as part of an operating system or a specific application, component, program, object, module, or sequence of instructions referred to as “computer programs.” The computer programs typically include one or more instructions set at various times in various memory and storage devices in a computer, and that, when read and executed by one or more processors in a computer, cause the computer to perform operations necessary to execute elements involving the various aspects.

A machine readable medium can be used to store software and data which when executed by a data processing system causes the system to perform various methods. The executable software and data may be stored in various places including, for example, ROM, volatile RAM, non-volatile memory and/or cache. Portions of this software and/or data may be stored in any one of these storage devices. Further, the data and instructions can be obtained from centralized servers or peer to peer networks. Different portions of the data and instructions can be obtained from different centralized servers and/or peer to peer networks at different times and in different communication sessions or in a same communication session. The data and instructions can be obtained in entirety prior to the execution of the applications. Alternatively, portions of the data and instructions can be obtained dynamically, just in time, when needed for execution. Thus, it is not required that the data and instructions be on a machine readable medium in entirety at a particular instance of time.

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., compact disk read-only memory (CD ROMS), digital versatile disks (DVDs), etc.), among others. The computer-readable media may store the instructions.

The instructions may also be embodied in digital and analog communication links for electrical, optical, acoustical, or other forms of propagated signals, such as carrier waves, infrared signals, digital signals, etc. However, propagated signals, such as carrier waves, infrared signals, digital signals, etc. are not tangible machine readable medium and are not configured to store instructions.

In general, a machine readable medium includes any mechanism that provides (i.e., stores and/or transmits) information in a form accessible by a machine (e.g., a computer, a network device, a personal digital assistant, a manufacturing tool, any device with a set of one or more processors, etc.).

In various embodiments, hardwired circuitry may be used in combination with software instructions to implement the techniques. Thus, the techniques are neither limited to any specific combination of hardware circuitry and software nor to any particular source for the instructions executed by the data processing system.

The description and drawings are illustrative and are not to be construed as limiting. The present disclosure is illustrative of inventive features to enable a person skilled in the art to make and use the techniques. Various features, as described herein, should be used in compliance with all current and future rules, laws and regulations related to privacy, security, permission, consent, authorization, and others. Numerous specific details are described to provide a thorough understanding. However, in certain instances, well known or conventional details are not described in order to avoid obscuring the description. References to one or an embodiment in the present disclosure are not necessarily references to the same embodiment; and, such references mean at least one.

The use of headings herein is merely provided for ease of reference, and shall not be interpreted in any way to limit this disclosure or the following claims.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, and are not necessarily all referring to separate or alternative embodiments mutually exclusive of other embodiments. Moreover, various features are described which may be exhibited by one embodiment and not by others. Similarly, various requirements are described which may be requirements for one embodiment but not other embodiments. Unless excluded by explicit description and/or apparent incompatibility, any combination of various features described in this description is also included here. For example, the features described above in connection with “in one embodiment” or “in some embodiments” can be all optionally included in one implementation, except where the dependency of certain features on other features, as apparent from the description, may limit the options of excluding selected features from the implementation, and incompatibility of certain features with other features, as apparent from the description, may limit the options of including selected features together in the implementation.

In the foregoing specification, the disclosure has been described with reference to specific exemplary embodiments thereof. It will be evident that various modifications may be made thereto without departing from the broader spirit and scope as set forth in the following claims. The specification and drawings are, accordingly, to be regarded in an illustrative sense rather than a restrictive sense.

What is claimed is:

1. A computer-implemented method for monitoring individuals in an environment to determine whether the individuals are at risk for infection, the computer-implemented method comprising:
receiving, with at least one processor, data associated with one or more measurements obtained by one or more sensors of a sensor band;
determining, with at least one processor, whether the one or more measurements obtained by the one or more sensors of the sensor band satisfies one or more conditions of a non-standard state, wherein the non-standard state is associated with an individual at risk for infection; and
providing, with at least one processor, an indication that an individual associated with the sensor band is at risk for infection.

2. The computer-implemented method of claim 1, wherein receiving the data associated with the one or more measurements obtained by the one or more sensors of the sensor band comprises:
receiving the data associated with the one or more measurements obtained by the one or more sensors associated with the individual based on the sensor band transmitting the data associated with the one or more measurements obtained by the one or more sensors,
wherein the sensor band transmits the data associated with the one or more measurements obtained by the one or more sensors by advertising the data associated with the one or more measurements to at least one gateway device.

3. The computer-implemented method of claim 2, wherein transmitting the data associated with the one or more measurements obtained by the one or more sensors of the sensor band by advertising the data associated with the one or more measurements to at least one gateway device comprises:
transmitting the data associated with the one or more measurements obtained by the one or more sensors of the sensor band by advertising the data associated with the one or more measurements from a Bluetooth low energy peripheral device included in the sensor band to the at least one gateway device.

4. The computer-implemented method of claim 1, wherein receiving the data associated with the one or more measurements obtained by the one or more sensors of the sensor band comprises:
receiving data associated with a temperature measurement of the individual; and
receiving data associated with a temperature measurement of an environment in which the individual is located, and
wherein determining whether the one or more measurements obtained by the one or more sensors of the sensor band satisfies one or more conditions of a non-standard state comprises:
determining a core temperature of the individual based on the temperature measurement of the individual and the temperature measurement of the environment in which the individual is located;
comparing the core temperature of the individual to a predetermined range of core temperatures corresponding to a high-temperature condition included in the one or more conditions of the non-standard state; and
determining that the core temperature of the individual satisfies a predetermined range of core temperatures corresponding to the high-temperature condition included in the one or more conditions of the non-standard state, and
wherein providing the indication that the individual is at risk for infection comprises:
providing the indication that the individual is at risk for infection based on determining that the core temperature of the individual satisfies the predetermined range of core temperatures corresponding to the high-temperature condition included in the one or more conditions of the non-standard state.

5. The computer-implemented method of claim 1, further comprising:
receiving data associated with registration of the one or more sensors from the sensor band;
registering the sensor band with the individual; and
generating a profile corresponding to the individual based on the data associated with the one or more measurements obtained by one or more sensors of the sensor band.

6. The computer-implemented method of claim 1, further comprising:
determining that the one or more measurements obtained by the one or more sensors of the sensor band represents one or more trends; and
determining whether the one or more trends indicate that the individual is at risk for infection,
wherein providing the indication that the individual associated with the sensor band is at risk for infection comprises:
providing the indication that the individual associated with the sensor band is at risk for infection based on determining that the trend indicates that the individual is at risk for infection.

7. The computer-implemented method of claim 5, further comprising:
determining that the one or more measurements obtained by the one or more sensors of the sensor band represents one or more trends; and
comparing the one or more trends to the profile corresponding to the individual; and
determining whether the one or more trends indicate that the individual is at risk for infection based on comparing the one or more trends to the profile corresponding to the individual,
wherein providing the indication that the individual associated with the sensor band is at risk for infection comprises:
providing the indication that the individual associated with the sensor band is at risk for infection based on determining that the one or more trends indicate that the individual is at risk for infection based on comparing the one or more trends to the profile corresponding to the individual.
